(19) United States
(12) Patent Application Publication
(10) Pub. No.: US 2020/0199170 A1
(43) Pub. Date: Jun. 25, 2020

(54) COMPOUNDS HAVING ANTI-INFLAMMATORY AND ANTI-OXIDANT ACTIVITY

(71) Applicant: UNIVERSITÀ DEGLI STUDI DI FERRARA, Ferrara (IT)

(72) Inventors: Paola PEDRINI, Ferrara (IT); Sergio FERRO, Ferrara (IT)

(21) Appl. No.: 15/751,367

(22) PCT Filed: Aug. 31, 2016
(86) PCT No.: PCT/EP2016/070453

§ 371 Date: Feb. 8, 2018

(30) Foreign Application Priority Data
Sep. 3, 2015 (IT) 102015000048373

(51) Int. Cl. C07J 7/00; A61K 8/63; A61K 9/08; A61Q 19/08; A61P 37/06; A61P 39/06; A61P 25/28; C12P 33/00

(52) U.S. Cl. CPC C07J 7/009; A61K 8/63; A61K 9/08; C12P 33/00; A61P 37/06; A61P 39/06; A61P 25/28; A61Q 19/08

(57) ABSTRACT

The present invention relates to new compounds of formula (1) wherein R is —C(═O)CH₂OH or —CH(OH)CH₂OH; R₁, is ═O or OH; R₂ is H or OH; R₃ is H or C₁-C₄alkyl; R₄ is H or C₁C₄alkyl. These compounds are obtained by a process using at least one bacterium belonging to the Actinobacteria class, preferably belonging to the Rhodococcus genus. Thanks to their antiinflammatory and anti-oxidant properties, the compounds of the invention can be advantageously used in the treatment of inflammatory diseases, autoimmune diseases and of diseases in need of a joined anti-inflammatory and anti-oxidant activity.

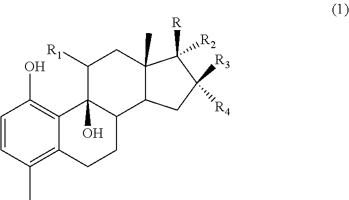

### FIELD OF THE INVENTION

The present invention relates to the field of pharmaceuticals.

In particular, it relates to new compounds of the steroid type having anti-inflammatory and anti-oxidant activity.

### BACKGROUND OF THE INVENTION

Corticosteroids are steroid hormones that are produced by the adrenal cortex. They are involved in many physiological mechanisms and, on the basis of their function, they are divided in three families, glucocorticoids—so-called as a result of their importance in the metabolism of glucose—mineralocorticoids—active in the balance of mineral salts, in particular sodium and potassium—and sexual hormones. [G. Faglia “Malattie del sistema endocrino e del metabolismo” (Diseases of the endocrine system and metabolism). McGraw-Hill Companies, 2002].

Glucocorticoids, whose main representative in humans is cortisol, are important in anti-inflammatory and immunosuppressive therapy [R. Newton, “Molecular mechanisms of glucocorticoid action: what is important?”, Thorax, 2000, 55:603-613]. They are widely used in the treatment of allergic reactions, inflammatory and autoimmune diseases, such as, for example, asthma, dermatitis, intestinal inflammatory illnesses, rheumatoid arthritis and multiple sclerosis, as well as in the prevention of rejection of transplanted organs and in the treatment of haematological neoplasia [P. J. Barnes, “Molecular mechanisms and cellular effects of glucocorticosteroids”, Immunol. Allergy Clin. North Am., 2005, 25:451-468; P. J. Barnes, I. Adcock, “Anti-inflammatory actions of steroids: molecular mechanisms”, Trends Pharmacol. Sci., 1993, 14(12):436-441; T. R. Cupps, A. S. Fauci, “Corticosteroid-mediated immuno-regulation in man”, Immunol. Rev., 1982, 65:133-155].

Unfortunately, the use of such pharmaceuticals (also known as corticosteroids or cortisone preparations) has a wide range of side effects. For this reason, and considering also the benefits and costs of the therapy, they are generally prescribed only in the presence of a severe pathology, when the latter cannot be overcome with the administration of other pharmaceuticals with a similar activity.

Furthermore, glucocorticoids have important interactions with other pharmaceuticals: the appearance of those side effects is even more probable when increasing doses and duration of the treatment; they are therefore more common in the case of a systemic therapy (oral or injected) and rare for local therapies (with creams, ointments and eye lotions), where they are generally used at low doses.

Moreover, those compounds, in particular cortisol and cortisone, are insoluble in water and this limits their use in therapy.

In order to produce pharmaceuticals having a greater therapeutic activity and fewer side effects, synthetic compounds that are structurally derived from progesterone (Scheme 1) and which differ from each other for minimal modifications in the structure have been studied.

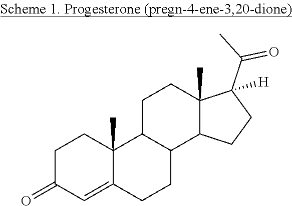 [SMILES: CC(=O)[C@H]1CCC2C3CCC4=CC(=O)CC[C@]4(C)C3CC[C@@]21C]

It is known that even minimal structural differences of these molecules can significantly modify the strength, the duration of action and the corticoid activity.

For example, WO200185755 and WO2002092100 describe steroid components having a cell modulation activity and anti-proliferative and anti-angiogenic effects. Those compounds are characterized mainly by the presence of a substituent group in position 2. They are obtained by means of conventional chemical synthesis processes. Additional steroid compounds are disclosed in: L. J. Chinn, “The preparation and pharmacology of some 11.beta.hydroxy-4-methylestratrienes”, J. Med. Chem., 1968, 11(4): 902-904; DE1138766B; chemical abstracts service. Columbus. Ohio. US, Raff. Thorsten et al: “Alteration of NF-.kappa.B expression by low doses of scavestrogens in peripheral blood mononuclear cells stimulated by LPS and sepsis”. XP002754923. Retrieved from STN Database accession No. 2001:899588; R. Shukla, P. K. Mehrotra, A. Dwivedi, V. P. Kamboj, “Pregnane derivatives as pregnancy interceptive agents: efficacy determination on growing trophoblasts (in vitro) and in pregnant hamsters (in vivo)”, Contraception, 1992, 45(6): 605-615.

In light of the above, steroid compounds are still sought, in particular having an anti-inflammatory action, but without the disadvantages of the known compounds, in particular cortisone compounds.

In particular, steroid compounds, which are effective and at the same time characterized by reduced harmful side effects, and that can also be used in therapeutic situations where it is necessary or preferable to use a water-soluble compound are still sought.

Steroids which carry a ketone group at C₁₁ (such as cortisone—Scheme 2, A1—and prednisone—Scheme 2, A2) are metabolically converted into molecules having a hydroxyl function at C₁₁ before being able to exert their activity (cortisol—Scheme 2, B1—and prednisolone—Scheme 2, B2—are obtained, respectively).

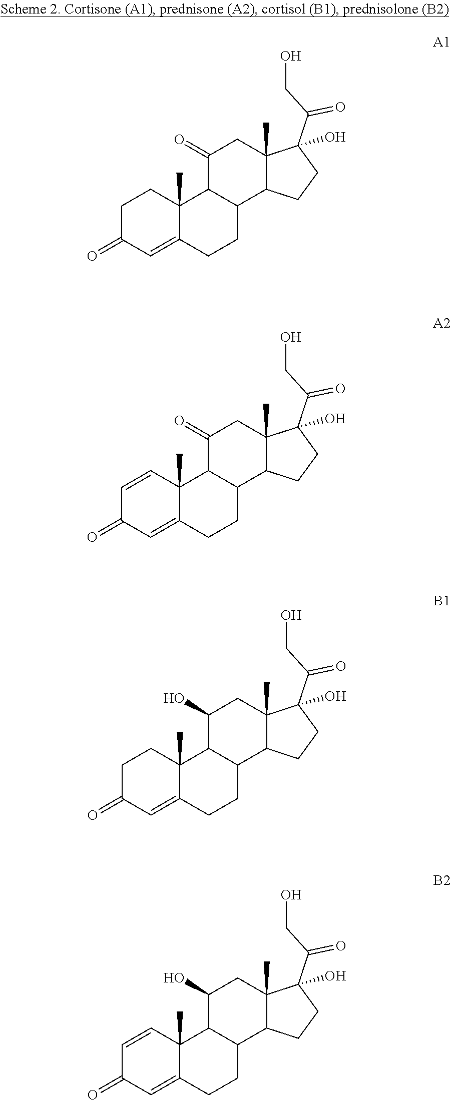 [SMILES: C[C@]12CCC(=O)C=C1CCC1C2C(=O)C[C@@]2(C)C1CC[C@]2(O)C(=O)CO ; C[C@]12C=CC(=O)C=C1CCC1C2C(=O)C[C@@]2(C)C1CC[C@]2(O)C(=O)CO ; C[C@]12CCC(=O)C=C1CCC1C2[C@@H](O)C[C@@]2(C)C1CC[C@]2(O)C(=O)CO ; C[C@]12C=CC(=O)C=C1CCC1C2[C@@H](O)C[C@@]2(C)C1CC[C@]2(O)C(=O)CO]

In this field, the transformations operated by microorganisms are an effective tool for the preparation of compounds that are otherwise difficult to obtain with conventional synthetic methods. The production of steroids and hormones is one of the best examples of successful application of the microbial technology in large-scale industrial processes [W. Charney, L. H. Herzog “Microbial transformations of steroids”, New York: Academic Press, 1976. pp. 5-73]. Research activities in this field started in the 1950s, following the identification of the pharmacological effects of cortisol and progesterone, and with the identification of the enzyme activity of hydroxylation at C₁₁ exerted by a microbial species such as Rhizopus [J. A. Hogg, “Steroids, the steroid community, and Upjohn in perspective: a profile of innovation”, Steroids, 1992, 57(12): 593-616]. Since then, multiple examples of microbial bioconversion of steroids and sterols have been reported in literature. These bio-transformations, for the most part connected with chemical synthesis steps, have provided suitable tools for the large-scale production of analogues of natural or modified steroids [P. Fernandes, A. Cruz, B. Angelova, H. M. Pinheiro, J. M. S. Cabral, “Microbial conversion of steroid compounds: recent developments”, Enzyme and Microbial Technology, 2003, 32(6): 688-705; H. N. Bhatti, R. A. Khera, “Biological transformations of steroidal compounds: a review”, Steroids, 2012, 77(12): 1267-1290].

The ability of some microorganisms to produce different metabolites, which cannot readily be obtained by chemical transformation, has proven to be an indispensable tool for the pharmaceutical industry [R. J. P. Cannell, A. R. Knaggs, M. J. Dawson, G. R. Manchee, P. J. Eddershaw, I. Waterhouse, P. R. Sutherland, G. D. Bowers, P. J. Sidebottom, “Microbial biotransformation of the angiotensin II antagonist GR117289 by Streptomyces rimosus to identify a mammalian metabolite”, Drug Metab. Dispos., 1995, 23(7): 724-729; C. Moussa, P. Houziaux, B. Danree, R. Azerad, “Microbial Models of Mammalian Metabolism: Fungal Metabolism of Phenolic and Nonphenolicp-Cymene-Related Drugs and Prodrugs. I. Metabolites of Thymoxamine”, Drug Metab. Dispos., 1997, 25(3): 301-310; E. A. Abourashed, A. M. Clark, C. D. Hufford, “Microbial models of mammalian metabolism of xenobiotics: An updated review”, Curr. Med. Chem., 1999, 6(5): 359-374].

In order to maximize the efficiency of the reaction, selected strains of a given microorganism are generally modified to optimize the expression of the genes of interest and/or to block specific metabolic pathways, in order to allow the accumulation of the intermediate products.

The actinobacteria (also referred to as actinomycetes or mycobacteria, because they are similar to filamentary mycetes) are a group of gram-positive bacteria (with the sole exception of Actinoplanes) comprising five sub-classes and 35 families [E. Stackebrandt, F. A. Rainey, N. L. Ward-Rainey, “Proposal for a new hierarchic classification system, Actinobacteria classis nov.”, Int. J. Syst. Bacteriol., 1997, 47:479-491].

These bacteria are mostly aerobic, unicellular, and live in branched filamentous colonies, in the ground or on the body of animals and humans. They use a large amount of products as sources of energy and also metabolize cellulose, fats, hydrocarbons, benzolic compounds and, to a lesser extent, lignins, tannins and rubbers.

The actinomycetes of greater medical and commercial/industrial relevance can be separated into anaerobic actinomycetes (genus Actinomyces [family Actynomicetaceae], which includes gram-positive bacilli, not acid-resistant, strictly or optional anaerobic, asporigens) and in aerobic actinomycetes (genera Nocardia, Rhodococcus [family Nocardiaceae], Actinomadura and Streptomyces [families Thermomonosporaceae and Streptomycetaceae]) including aerobic gram-positive bacilli, which are partially acid-resistant.

The genus Rhodococcus includes aerobic bacteria species that are not mobile and do not produce spores. Though some species are pathogens, the majority of them are benign and capable of prospering in a vast variety of environments, including soil, water and eukaryotic cells. The strains of Rhodococcus are important for their ability to metabolize a wide range of bioactive compounds, thus producing steroids, acrylamides and acrylic acid, and also for their involvement in the biodesulphurization processes of fossil fuels. Another important application of Rhodococcus can be identified in the bioconversion of noxious environmental pollutants, including toluene, naphthalene, herbicides, polychlorobiphenyls and even trinitrotoluene, and they are therefore widely studied in the field of applied microbiology. Furthermore, it is known that various species of Rhodococcus are able to degrade natural phytosterols: steroids are intermediate products of this reaction.

The actinobacteria are known for degrading steroid compounds. However, any degradation process is different and its outcome is hardly predictable a priori. Very often, it is necessary to genetically modify the bacterial strain in order to obtain the compound of interest, and this requires elaborate and expensive procedures.

### SUMMARY OF THE INVENTION

It has now surprisingly been found that bacteria belonging to the class of actinobacteria are able to transform some known steroid compounds into new compounds, which possess an anti-inflammatory and anti-oxidant activity, have fewer side effects and a greater therapeutic potential in comparison to known compounds, in particular to the compounds from which they are produced.

Such new compounds are obtained by means of a microbiological fermentation process without any need for particular chemical reagents or complicated and expensive process conditions.

Said process is also less polluting and can readily be used on a large scale.

Therefore, the present invention relates to a compound having the following formula (1)

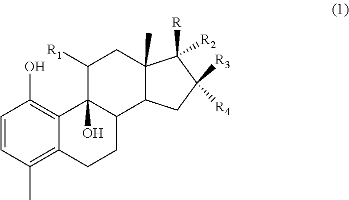

wherein

R is —C(═O)CH₂OH or —CH(OH)CH₂OH;

R₁ is ═O or OH;

R₂ is H or OH;

R₃ is H or C₁-C₄alkyl;

R₄ is H or C₁-C₄alkyl.

Preferred compounds according to the present invention are the following:

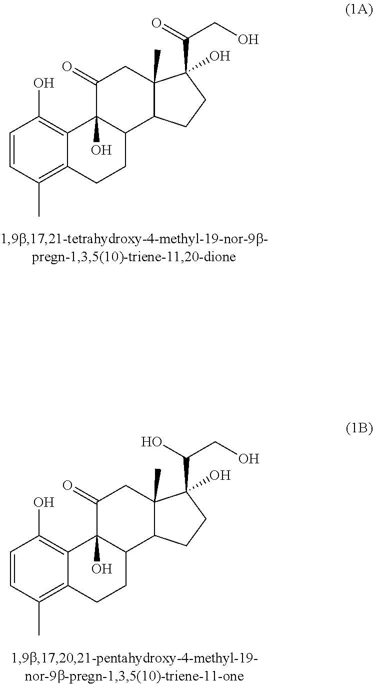

The present invention further relates to a process for obtaining the compounds having formula (1), which uses at least one bacterium belonging to the class of Actinobacteria.

Preferably, said bacterium belongs to the genus Rhodococcus.

Said process has the advantage of being a fermentation process, which uses aerobic and non-pathogenic bacteria species. Furthermore, the bacteria species used are wild type, therefore they do not require genetic modifications, thereby avoiding the ethical problems and regulatory requirements connected with their potential genetic manipulations. This allows the process also to be readily used on a large scale.

Furthermore, such a process has a low level of environmental pollution because it is carried out in an aqueous culture medium and at substantially room temperature, hence without any need for energy expenditure or particular chemical reagents or reaction conditions.

The process of the invention for obtaining the compounds having formula (1) comprises the administration of compounds characterized by the following formula (2) to a bacterial culture comprising at least one bacterium belonging to the class of Actinobacteria:

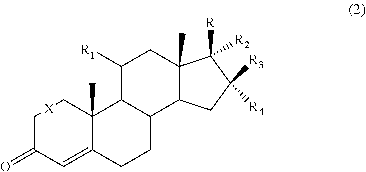

wherein

R, R₁, R₂, R₃ and R₄ have the meanings set out above for the compounds having formula (1) and

X indicates a single or double bond.

The new compounds of the present invention (compounds having formula (1)) have an anti-inflammatory, antioxidant and neuroprotective activity. Because of the joint presence of those activities, they have a better therapeutic potential than known steroid compounds, for example cortisone, in particular in comparison to the activity of the compounds from which they are derived (compounds having formula (2)).

In particular, they exhibit a significant reduction of the harmful side effects that are produced by the known compounds, especially when they are administered in massive and/or prolonged doses.

Therefore, it is also an object of the present invention the use of the compound having formula (1) as a medicament.

In particular, it is an object of the present invention the use of the compound having formula (1) in the treatment of inflammatory diseases, autoimmune diseases and diseases that require joint anti-inflammatory and antioxidant activities.

It is particularly preferred the use of the compounds having formula (1) in the treatment of degenerative diseases of neuronal cells, such as Alzheimer's disease and Parkinson's disease.

Furthermore, the new compounds having formula (1), unlike the known compounds, are soluble in water. This allows the use of such compounds in therapies that cannot be treated by the known steroidal compounds, for example, cortisone and cortisol, because of their insolubility in water.

The present invention also relates to a pharmaceutical composition comprising the compounds of formula (1) together with at least one suitable pharmaceutically acceptable vehicle or excipient.

In a preferred embodiment, said pharmaceutical composition is in aqueous formulation.

### DETAILED DESCRIPTION OF THE INVENTION

### Figures

FIG. 1: FIG. 1A shows the measured concentrations of IL-1α following the treatment of keratinocytes with dimethyl sulphoxide (DMSO), with TNF-α only, and with TNF-α following a pre-treatment with 1A (10 μM solution), or 1B (10 μM solution), or with an admixture thereof (10 μM and 20 μM solution). FIG. 1B shows the results of real-time PCR measurements, which are intended to quantify the gene expression. The “control” is the response of the non-stimulated cells (absence of inflammatory process), “TNF-α” is the response of the cells stimulated with cytokine, while “1A+TNF-α” and “1B+TNF-α” are the responses obtained with the cells pre-treated with 1A and 1B, respectively.

FIG. 2: it illustrates the antioxidant activity of the compounds 1A and 1B in comparison with compounds having a known antioxidant activity, that are ascorbic acid, Trolox and Quercetin.

FIG. 3: it shows the analysis of cell viability in neuronal cells following a treatment with glutamate (Glu), prior exposure to betamethasone (Beta) or pre-treatment with the compounds having formula 1A and 1B.

The compounds having formula (1) are obtained by means of a process that comprises the use of bacteria belonging to the class of Actinobacteria.

The Actinobacteria (also referred to as Actinomycetes or Mycobacteria) are a group of gram-positive bacteria (with the sole exception of Actinoplanes) which comprises five sub-classes (Acidimicrobidae, Actinobacteridae, Coriobacteridae, Rubrobacteridae, Sphaerobacteridae) and 35 families (Acidimicrobiaceae, Rubrobacteraceae, Coriobacteriaceae, Sphaerobacteraceae, Actinomycetaceae, Propionibacteriaceae, Nocardioidaceae, Micrococcaceae, Cellulomonadaceae, Promicromonosporaceae, Dermatophilaceae, Brevibacteriaceae, Dermabacteraceae, Intrasporangiaceae, Jonesiaceae, Microbacteriaceae, Corynebacteriaceae, Mycobacteriaceae, Nocardiaceae, Gordoniaceae, Tsukamurellaceae, Dietziaceae, Pseudonocardiaceae, Streptomycetaceae, Streptosporangiaceae, Nocardiopsaceae, Thermomonosporaceae, Micromonosporaceae, Frankiaceae, Geodermatophilaceae, Microsphaeraceae, Sporichthyaceae, Acidothermaceae, Glycomycetaceae, Bifidobacteriaceae) [E. Stackebrandt, F. A. Rainey, N. L. Ward-Rainey, Int. J. Syst. Bacteriol., 1997, 47:479-491].

A bacterium belonging to any of the mentioned sub-classes and families may be used in the present invention.

In particular, said bacterium may belong to any of the following sub-classes: Acidimicrobidae, Actinobacteridae, Coriobacteridae, Rubrobacteridae, Sphaerobacteridae.

Preferably, said bacterium belongs to a family selected from the group consisting of the families Nocardiaceae, Thermomonosporaceae and Streptomycetaceae.

Even more preferably, said bacterium belongs to one of the following genera: Nocardia and Rhodococcus (family Nocardiaceae); Actinomadura (family Thermomonosporaceae); Streptomyces (family Streptomycetaceae).

A particularly preferred genus is the genus Rhodococcus.

Within the genus Rhodococcus, it is particularly preferable to use bacteria that belong to one of the following species: R. aurantiacus, R. baikonurensis, R. boritolerans, R. equi, R. coprophilus, R. corynebacterioides (synonym: Nocardia corynebacterioides), R. erythropolis, R. fascians, R. globerulus, R. gordoniae, R. jostii, R. koreensis, R. kroppenstedtii, R. maanshanensis, R. marinonascens, R. opacus, R. percolatus, R. phenolicus, R. polyvorum, R. pyridinivorans, R. rhodochrous, R. rhodnii (synonym: Nocardia rhodnii), R. ruber (synonym: Streptothrix rubra), R. jostii RHA1, R. triatomae, R. tukisamuensis, R. wratislaviensis (synonym: Tsukamurella wratislaviensis), R. yunnanensis, R. zopfii.

The species Rhodococcus rhodnii is particularly preferred.

Said species comprises many different bacterial strains, which may all be used in the process of the invention.

In particular, preferred bacterial strains belonging to the species Rhodococcus rhodnii to be used in the process of the invention are the following: ATCC 35071, ATCC 35701, B/O, BCRC 13390, CCRC 13390, CCUG 23604, CECT 5750, CGMCC 4.1816, CIP 104181, DSM 43336, DSM 43337, DSM 43959, DSM 43960, Goodfellow N445, Hill B/O, Hill strain B/O, HillB/O, HillB/O ATCC35071, IEGM 555, IFM 149, IMSNU 21249, JCM 3203, KCC A-0203, KCCA-0203, KCTC 9805, LMG 5362, LMG 5363, N445, NBRC 100604, NCIB 11279, NCIMB 11279, NRRL B-16535, PCM 2157, RIA 1613, Ridell X58, Strain N445, VKM Ac-1187.

More preferably, said bacterium is from a strain selected from the group consisting of the following bacterial strains: ATCC 35071, DSM 43336, DSM 43337, DSM 43959 and DSM 43960.

All said bacterial strains are commonly available, in particular from the respective collections of international bacterial cultures.

According to the process of the invention, the bacteria are grown in a suitable culture medium generally for a period between 8 and 72 hours, preferably between 24 and 48 hours, preferably at a temperature between 25 and 30° C.

The culture medium may be any medium that is known in the sector for the growth of bacteria of the class Actinobacteria. For example, said medium may be one of the following: Columbia broth, Tryptic soy broth, Plate count broth or other broths with different amounts of carbon source.

As a preferred culture medium, the broth “Plate count broth” (PCB) may be used, which comprises tryptone, yeast extract and glucose.

It is also possible to produce a pre-inoculum of the bacterium, first growing the bacterium for a period of time in a suitable culture medium and subsequently transferring the entire culture (pre-inoculum) to another suitable sterile culture medium in which the bacterium is further grown under the described above conditions.

Subsequently, a compound having formula (2), as described above, is added in the culture medium of the bacterium.

Preferably, the compound having formula (2) is selected from the following known compounds:

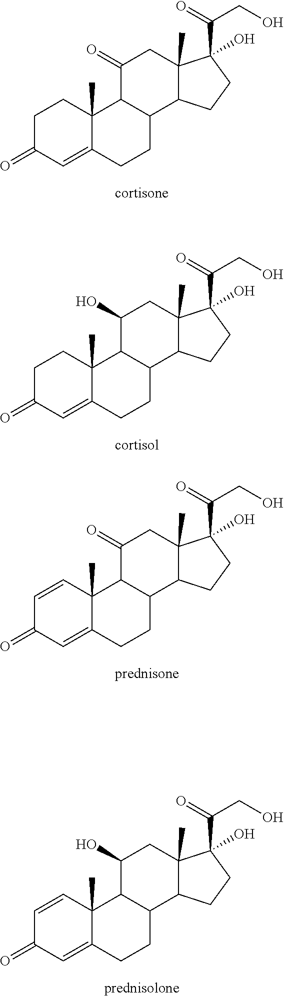 [SMILES: C[C@]12CCC(=O)C=C1CCC1C2C(=O)C[C@@]2(C)C1CC[C@]2(O)C(=O)CO ; C[C@]12CCC(=O)C=C1CCC1C2[C@@H](O)C[C@@]2(C)C1CC[C@]2(O)C(=O)CO ; C[C@]12C=CC(=O)C=C1CCC1C2C(=O)C[C@@]2(C)C1CC[C@]2(O)C(=O)CO ; C[C@]12C=CC(=O)C=C1CCC1C2[C@@H](O)C[C@@]2(C)C1CC[C@]2(O)C(=O)CO]

Even more preferably, said compound is selected from prednisone and cortisone.

The compound having formula (2) is added to the culture medium at a concentration preferably comprised between 0.5 grams/litre and 10.0 grams/litre, more preferably between 1 gram/litre and 5 grams/litre.

The culture is then left in fermentation for a variable time, typically from 8 to 96 hours, preferably from 24 to 48 hours.

The compounds having formula (1) are thereby obtained.

The products may then be extracted and purified from the culture medium by means of the normal extraction and purification methods that are known in the field.

For example, the culture may be centrifuged in order to eliminate the cells; subsequently, the supernatant may be extracted with suitable organic solvents.

The final products may then be separated, for example, by means of chromatography.

The specific compound having formula (2), added to the culture medium, is selected on the basis of the compound having formula (1) that one wants to get.

In a particular embodiment, in order to obtain a compound having formula (1A), the compound having formula (2) may be selected from: cortisone, cortisol, prednisone and prednisolone. Preferably, it is selected from cortisone and prednisone.

In another particular embodiment, in order to obtain a compound having formula (1B), the compound having formula (2) may be selected from cortisone and prednisone.

The person skilled in the art, as a result of his common knowledge in the field, will know how to select the compound having formula (2) to be administered to the bacteria on the basis of the final compound having formula (1) that it is desirable to obtain.

The production process of the new compounds having formula (1) is a fermentation process, which can readily be carried out on a large scale because the microorganism used is aerobic and non-pathogenic.

From the point of view of the environment, the fermentation process of the growth of the microorganism and the biotransformation of the compounds having the general formula (2) is a “green” process, in fact it is generally carried out in an aqueous culture broth and at room temperature.

With suitable arrangements, which the person skilled in the art can carry out as a result of his general knowledge in the field, the above-described process, which is particularly suitable for applications on a small scale, can readily be transferred to a large scale. Therefore, it can advantageously be used industrially.

The compounds having formula (1) have an anti-inflammatory activity comparable with that of betamethasone, an antioxidant activity comparable with that of ascorbic acid and a neuro-protective activity.

Furthermore, they are neither cytotoxic nor mutagenic.

Therefore, the present invention relates also to the use of compounds having the general formula (1) as medicaments.

In particular, their use in the treatment of diseases wherein it is desired to have an anti-inflammatory and/or anti-oxidant and/or neuro-protective action is preferred.

For example, they can be used in the treatment of inflammatory diseases or in any disease in which an abnormal inflammation is present, such as allergic reactions, myopathies, cancer, atherosclerosis, ischaemic heart disease.

Also, they can be used in the treatment of autoimmune diseases, i.e. of any pathological state arising from an abnormal immune response of the body to substances and tissues that are normally present in the body.

For example, they can be used in the treatment of inflammatory and/or autoimmune diseases, such as asthma, dermatitis, intestinal inflammatory diseases, rheumatoid arthritis, multiple sclerosis, dermatological diseases.

They can be used also in the treatment of diseases resulting from allergic reactions or in the immunosuppressive therapy.

They can be used also in the prevention of the rejection of transplants.

Thanks to their anti-oxidant activity, they can also be advantageously used in any disease or condition wherein an anti-oxidant effect is desired. Also, they can be used for the prevention of diseases, such as cardiovascular diseases.

They are also particularly useful in the treatment of diseases wherein a joined anti-inflammatory and anti-oxidant activity is desired.

Furthermore, in view of their combined anti-inflammatory and antioxidant action, they can be advantageously used in the treatment of degenerative diseases of the neuronal cells, such as, for example, Alzheimer's and Parkinson's diseases.

The compounds having formula (1) can be administered as a medicament to a subject in need thereof by means of conventional methods.

Advantageously, they can be administered via the parenteral route, the oral route, the rectal route or by inhalation, but other forms may also be suitable. The person skilled in the art is able to decide the administration timings based on the conditions of the patient, the gravity of the pathology, the response of the patient and other chemical parameters.

The compounds having formula (1) may be included in a pharmaceutical composition.

Therefore, the present invention also relates to a pharmaceutical composition that comprises the compounds having formula (1) together with a suitable vehicle.

The average amounts of the compound having formula (1) may vary depending on the conditions of the patient, the gravity of the pathological condition and other variables. In particular, they would have to be established on the basis of the prescriptions and the indications of a qualified doctor.

The pharmaceutical composition contains, together with the active ingredient, at least one pharmaceutically acceptable vehicle or excipient. For example, it may contain particularly advantageous formulation co-adjuvants, such as solubilizing agents, dispersion agents, suspension agents and emulsifying agents.

The compositions may be administered via the oral route, for example, in solid or liquid form. They may have any advantageous form, for example, compresses, coated compresses, capsules, aqueous or oily suspensions, solutions, emulsions, syrups or dried products.

As a result of their anti-inflammatory and antioxidant properties, the new compounds of formula (1) can also be used in the cosmetic field, for example, in the field of skin care. In particular, they can be used to prevent the ageing of the skin of the body and face.

For example, they can be used in products such as creams, lotions, oils, gels, hydrating milks and sun creams.

The present invention will now be further illustrated by the following examples.

### EXAMPLES

### Example 1

Production of the Compounds having Formula 1A and 1B by Administration of Cortisone to the Strain Rhodococcus rhodnii DSM 43336

The strain Rhodococcus rhodnii DSM 43336 is inoculated in 10 mL of PCB broth [Plate Count Broth] which is constituted by tryptone (5 g/L), yeast extract (2.5 g/L) and glucose (1 g/L), which has been previously sterilized. After 48 hours of growth under agitation (100-110 rpm) at 26° C., the entire culture (pre-inoculum) is transferred into a 500 mL flask containing 200 mL of sterile PCB broth. After 72 hours of growth at 26° C. under agitation (100-110 rpm), 2 mL of cortisone solution dissolved in DMSO [dimethyl sulphoxide] (100 mg/mL), in order to obtain a concentration of 1 g/L, are added. After 24 hours of biotransformation, the culture is centrifuged in order to eliminate the cells (6000 rpm). The supernatant is extracted with ethyl acetate (3×200 mL), dehydrated with anhydrous Na₂SO₄ and, after filtration and evaporation of the solvent, the products are separated by chromatography (stationary phase: silica; eluent: ethyl acetate): the compounds having formula 1A (55%) and 1B (30%) are obtained.

### Example 2

Production of the Compounds having Formula 1A and 1B by Administration of Cortisone to the Strain Rhodococcus rhodnii DSM 43336

The strain Rhodococcus rhodnii DSM 43336 is inoculated in 10 mL of PCB broth which is constituted by tryptone (5 g/L), yeast extract (2.5 g/L) and glucose (1 g/L), which has been previously sterilized. After 48 hours of growth under agitation (100-110 rpm) at 26° C., the entire culture (pre-inoculum) is transferred into a 500 mL flask containing 200 mL of sterile PCB broth. After 72 hours of growth at 30° C. under agitation (100-110 rpm), 2 mL of cortisone solution dissolved in DMSO (100 mg/mL), in order to obtain a concentration of 1 g/L, are added. After 36 hours of biotransformation, the culture is centrifuged in order to eliminate the cells (6000 rpm). The supernatant is extracted with ethyl acetate (3×200 mL), dehydrated with anhydrous Na₂SO₄ and, after filtration and evaporation of the solvent, the products are separated by chromatography (stationary phase: silica; eluent: ethyl acetate): the compounds having formula 1A (50%) and 1B (25%) are obtained.

### Example 3

Production of the Compounds having Formula 1A and 1B by Administration of Cortisone to the Strain Rhodococcus rhodnii DSM 43960

The strain Rhodococcus rhodnii DSM 43960 is inoculated in 10 mL of PCB broth which is constituted by tryptone (5 g/L), yeast extract (2.5 g/L) and glucose (1 g/L), which has been previously sterilized. After 48 hours of growth under agitation (100-110 rpm) at 26° C., the entire culture (pre-inoculum) is transferred into a 500 mL flask containing 200 mL of sterile PCB broth. After 72 hours of growth at 26° C. under agitation (100-110 rpm), 2 mL of cortisone solution dissolved in DMSO (100 mg/mL), in order to obtain a concentration of 1 g/L, are added. After 36 hours of biotransformation, the culture is centrifuged in order to eliminate the cells (6000 rpm). The supernatant is extracted with ethyl acetate (3×200 mL), dehydrated with anhydrous Na₂SO₄ and, after filtration and evaporation of the solvent, the products are separated by chromatography (stationary phase: silica; eluent: ethyl acetate): the compounds having formula 1A (70%) and 1B (20%) are obtained.

### Example 4

Production of the Compound having Formula 1A by Administration of Cortisol to the Strain Rhodococcus rhodnii DSM 43336

The strain Rhodococcus rhodnii DSM 43336 is inoculated in 10 mL of PCB broth which is constituted by tryptone (5 g/L), yeast extract (2.5 g/L) and glucose (1 g/L), which has been previously sterilized. After 48 hours of growth under agitation (100-110 rpm) at 26° C., the entire culture (pre-inoculum) is transferred into a 500 mL flask containing 200 mL of sterile PCB broth. After 72 hours of growth at 26° C. under agitation (100-110 rpm), 2 mL of cortisol solution dissolved in DMSO (100 mg/mL), in order to obtain a concentration of 1 g/L, are added. After 4 days of biotransformation, the culture is centrifuged in order to eliminate the cells (6000 rpm). The supernatant is extracted with ethyl acetate (3×200 mL), dehydrated with anhydrous Na₂SO₄ and, after filtration and evaporation of the solvent, the products are separated by chromatography (stationary phase: silica; eluent: ethyl acetate): only the product having formula 1A (10%) is obtained.

### Example 5

Production of the Compounds having Formula 1A and 1B by Administration of Prednisone to the Strain Rhodococcus rhodnii DSM 43336

The strain Rhodococcus rhodnii DSM 43336 is inoculated in 10 mL of PCB broth which is constituted by tryptone (5 g/L), yeast extract (2.5 g/L) and glucose (1 g/L), which has been previously sterilized. After 48 hours of growth under agitation (100-110 rpm) at 26° C., the entire culture (pre-inoculum) is transferred into a 500 mL flask containing 200 mL of sterile PCB broth. After 72 hours of growth at 26° C. under agitation (100-110 rpm), 2 mL of prednisone solution dissolved in DMSO (100 mg/mL), in order to obtain a concentration of 1 g/L, are added. After 48 hours of biotransformation, the culture is centrifuged in order to eliminate the cells (6000 rpm). The supernatant is extracted with ethyl acetate (3×200 mL), dehydrated with anhydrous Na₂SO₄ and, after filtration and evaporation of the solvent, the products are separated by chromatography (stationary phase: silica; eluent: ethyl acetate): the compounds having formula 1A (50%) and 1B (25%) are obtained.

### Example 6

Production of the Compound having Formula 1A by Administration of Prednisolone to the Strain Rhodococcus rhodnii DSM 43336

The strain Rhodococcus rhodnii DSM 43336 is inoculated in 10 mL of PCB broth which is constituted by tryptone (5 g/L), yeast extract (2.5 g/L) and glucose (1 g/L), which has been previously sterilized. After 48 hours of growth under agitation (100-110 rpm) at 26° C., the entire culture (pre-inoculum) is transferred into a 500 mL flask containing 200 mL of sterile PCB broth. After 72 hours of growth at 26° C. under agitation (100-110 rpm), 2 mL of prednisolone solution dissolved in DMSO (100 mg/mL), in order to obtain a concentration of 1 g/L, are added. After 5 days of biotransformation, the culture is centrifuged in order to eliminate the cells (6000 rpm). The supernatant is extracted with ethyl acetate (3×200 mL), dehydrated with anhydrous Na₂SO₄ and, after filtration and evaporation of the solvent, the products are separated by chromatography (stationary phase: silica; eluent: ethyl acetate): only the compound having formula 1A is obtained (10%).

### Example 7

Activity Test in vitro of the Compounds having the Formula 1A and 1B

Anti-Inflammatory Activity

The anti-inflammatory properties of the new compounds having formula 1A and 1B have been tested.

The cells (human keratinocytes acquired from Life Technologies) have been pretreated for 24 hours with 1A or 1B and subsequently stimulated with cytokines, otherwise referred to as Tumor Necrosis Factor, TNF-α, in order to mimic an inflammatory process.

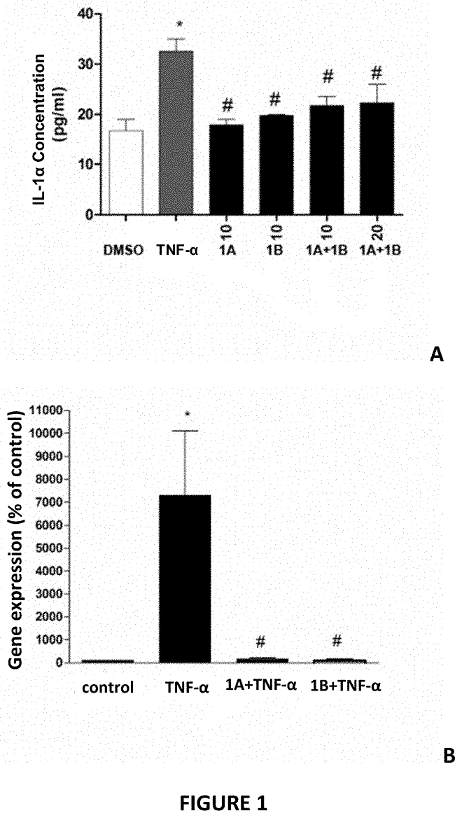

TNF-α induces inflammation in the cells treated, that is to say, the cell production of interleukin-1 (IL-1α). FIG. 1A shows the concentrations measured of IL-1α following the treatment of the keratinocytes with dimethyl sulphoxide (DMSO, solvent; it constitutes the negative control), with TNF-α alone, and with TNF-α after an initial pre-treatment with 1A (10 μM solution), or 1B (10 μM solution), or with an admixture thereof (10 μM and 20 μM solution). The data obtained show that the compounds having formula 1A and 1B possess an anti-inflammatory activity, since the cell production of IL-1α is comparable with that of the negative control.

The cell response has also been set out by means of real-time PCR measurements, which are intended to quantify the gene expression (the production of IL-1α is an activity that is codified by specific genes). In FIG. 1B, the so-called “control” is the response of the non-stimulated cells (absence of inflammatory process), “TNF-α” is the response of the cells stimulated with cytokine, while “1A+TNF-α” and “1B+TNF-α” are the responses obtained with the cells pre-treated with 1A and 1B, respectively. The absence of gene expression is an indication of the inhibition of the inflammatory response.

Antioxidant Activity

For the study of the antioxidant activity, use was made of a photochemiluminescence test that allows evaluating the antioxidant capacity of the tested molecules with respect to the superoxide radical (O₂⁻); the latter is photochemically generated by means of UV radiations. The marker used is luminol, a molecule that is capable of emitting chemiluminescence when it is oxidized by free radicals; such emission of light is then measured by means of a suitable instrument (Analityk Jena AG: Photochem®—Antioxidants and Free Radicals). The presence of antioxidant substances in the reaction admixture deactivates the radical species by inhibiting the emission of chemiluminescence. The analysis is very rapid and sensitive. Furthermore, by applying two different analytical protocols, referred to as ACW (Antioxidant Capacity Water soluble) and ACL (Antioxidant Capacity Lipid soluble), it is possible to estimate, for one and the same compound, the contributions to the total antioxidant capacity of both components, the water-soluble one and the fat-soluble one.

Since the antioxidant activity cannot be measured in absolute terms, the common practice is to compare the activity of a compound with that of molecules having an antioxidant capacity that is already known, such as, for example, tocopherol (otherwise known as vitamin E). By comparing the recorded values with the measurements relating to the standard reference molecules (ascorbic acid for the ACL protocol and Trolox for the ACW protocol), the antioxidant capacity of the product being examined is obtained.

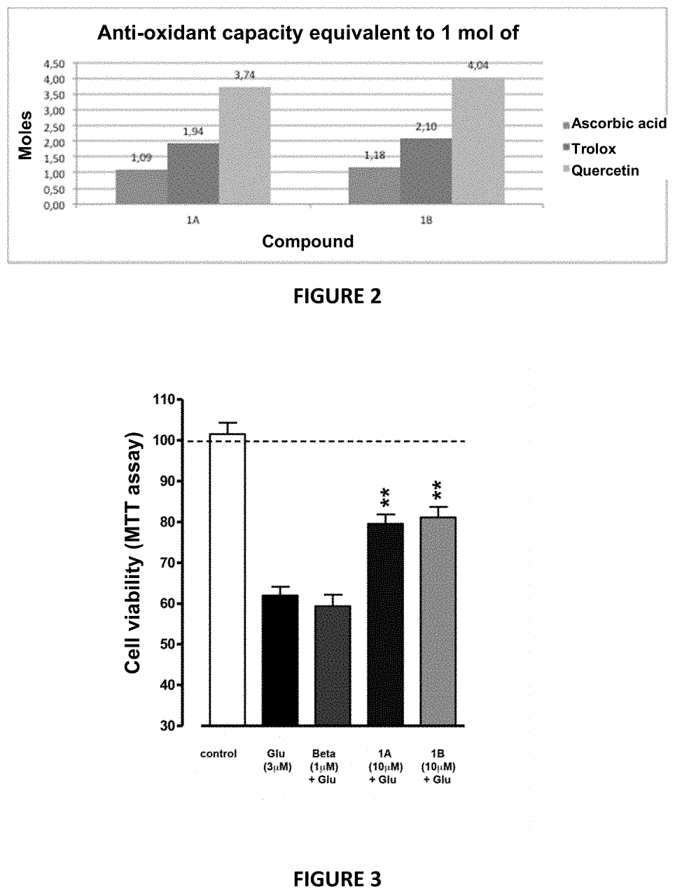

As shown in FIG. 2, the compounds having formula 1A and 1B have an antioxidant activity comparable to that of ascorbic acid (the graph shows that 1.09 moles of 1A and 1.18 moles of 1B have the antioxidant activity of 1 mole of ascorbic acid). By knowing the relationship that exists between the antioxidant activity of Trolox and Quercetin, it is also possible to relate the antioxidant activity of the product under investigation to the latter molecule.

Cell Viability

The compounds having formula 1A and 1B have been tested in order to determine the cell viability on lines of human keratinocytes HaCaT. The test has been carried out by exposing the cells HaCaT at different concentrations (from 5 to 500 μM) of the compounds under investigation (1A and 1B) for 24 hours at 37° C.; subsequently, the cell viability was measured by means of the MTT assay (3-(4,5-dimethylthiazol-2-yl)-2,5-diphenyltetrazole bromide).

For both the compounds under investigation, no cytotoxicity has been found, for none of the concentrations tested, since the viability of the keratinocytes treated does not decrease with respect to that of the control culture.

Mutagenic Activity

The compounds having formula 1A and 1B have been examined in order to investigate their possible mutagenic activity in accordance with the Ames test. The evaluations were carried out using the strains TA98 and TA100 of Salmonella typhimurium, both in the presence and in absence of metabolic activation (S9-Mix). The tests were carried out using solutions of the compounds having formula 1A and 1B starting from the respective stock solutions [100 mg/mL DMSO], which were subsequently diluted so as to have a spectrum of assay concentrations of between 100 and 10000 μg/plate. Each test was prepared with suitable controls both positive and negative.

The results obtained with the Ames test show that the compounds having formula 1A and 1B are not mutagenic under the conditions used in the study, at the concentrations considered.

Neuro-Protective Activity

Using again an MTT assay, it has also been found that the compounds having formula 1A and 1B have a neuro-protective activity on the death of neuronal cells caused by glutamate, when compared with betamethasone; the latter does not exert any neuro-protective effect and has therefore been used as a negative control.

FIG. 3 shows how the cell viability is significantly reduced following a treatment with glutamate (Glu); the preventive exposure to betamethasone (Beta) does not improve the result, while a pre-treatment with the compounds having formula 1A and 1B allows maintaining the viability of the cells at higher levels, near to 80%.

1. Compound of formula (1) 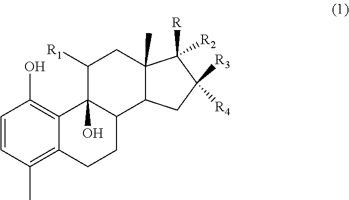
wherein
R is —C(═O)CH₂OH or —CH(OH)CH₂OH;
R₁ is ═O or OH;
R₂ is H or OH;
R₃ is H or C₁-C₄alkyl;
R₄ is H or C₁-C₄alkyl.

2. The compound according to claim 1, which is selected from the following compounds: 1,9β,17,21-tetrahydroxy-4-methyl-19-nor-9β-pregn-1,3,5(10)-triene-11,20-dione (1A) and 1,9β,17,20,21-pentahydroxy-4-methyl-19-nor-9β-pregn-1,3,5(10)-triene-11-one (1B).

3. A process for the obtainment of the compound of claim 1 or 2 comprising the step of administering to a bacterial culture comprising at least one bacterium belonging to the Actinobacteria class one or more compounds characterized by the following formula (2): 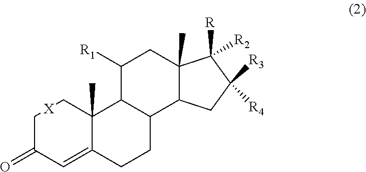
wherein
R, R₁, R₂, R₃ and R₄ have the meanings mentioned in claim 1, and
X is a single or double bond.

4. The process according to claim 3, wherein said bacterium belongs to a sub-class selected from the following sub-classes: Acidimicrobidae, Actinobacteridae, Coriobacteridae, Rubrobacteridae, and Sphaerobacteridae.

5. The process according to claim 4, wherein said bacterium belongs to a family selected from the following families: Acidimicrobiaceae, Rubrobacteraceae, Coriobacteriaceae, Sphaerobacteraceae, Actinomycetaceae, Propionibacteriaceae, Nocardioidaceae, Micrococcaceae, Cellulomonadaceae, Promicromonosporaceae, Dermatophilaceae, Brevibacteriaceae, Dermabacteraceae, Intrasporangiaceae, Jonesiaceae, Microbacteriaceae, Corynebacteriaceae, Mycobacteriaceae, Nocardiaceae, Gordoniaceae, Tsukamurellaceae, Dietziaceae, Pseudonocardiaceae, Streptomycetaceae, Streptosporangiaceae, Nocardiopsaceae, Thermomonosporaceae, Micromonosporaceae, Frankiaceae, Geodermatophilaceae, Microsphaeraceae, Sporichthyaceae, Acidothermaceae, Glycomycetaceae, and Bifidobacteriaceae.

6. The process according to claim 5, wherein said bacterium belongs to a genus selected from one of the following genera: Nocardia and Rhodococcus (Nocardiaceae family); Actinomadura (Thermomonosporaceae family); Streptomyces (Streptomycetaceae family).

7. The process according to claim 6, wherein said bacterium belongs to one of the following species: R. aurantiacus, R. baikonurensis, R. boritolerans, R. equi, R. coprophilus, R. corynebacterioides, R. erythropolis, R. fascians, R. globerulus, R. gordoniae, R. jostii, R. koreensis, R. kroppenstedtii, R. maanshanensis, R. marinonascens, R. opacus, R. percolatus, R. phenolicus, R. polyvorum, R. pyridinivorans, R. rhodochrous, R. rhodnii, R. ruber, R. jostii RHA1, R. triatomae, R. tukisamuensis, R. wratislaviensis (synonymous: Tsukamurella wratislaviensis), R. yunnanensis and R. zopfii.

8. The process according to claim 7, wherein said bacterium belongs to the species Rhodococcus rhodnii.

9. The process according to claim 8, wherein said bacterium is from a strain selected from the group consisting of the strains ATCC 35071, DSM 43336, DSM 43337, DSM 43959 and DSM 43960.

10. The process according to anyone of claims 3-9, wherein said compound of formula (2) is selected from the following compounds:

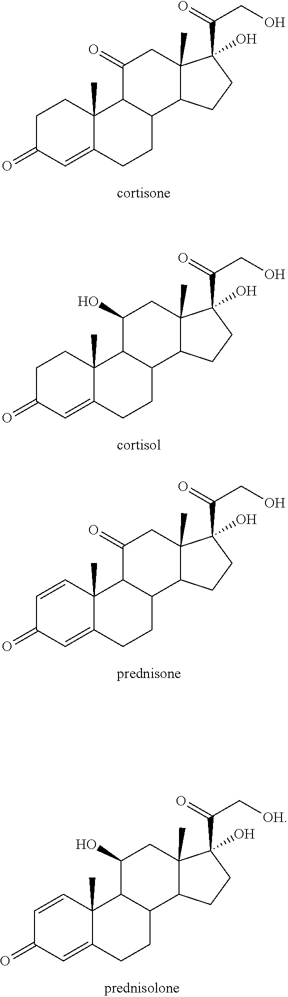 [SMILES: C[C@]12CCC(=O)C=C1CCC1C2C(=O)C[C@@]2(C)C1CC[C@]2(O)C(=O)CO ; C[C@]12CCC(=O)C=C1CCC1C2[C@@H](O)C[C@@]2(C)C1CC[C@]2(O)C(=O)CO ; C[C@]12C=CC(=O)C=C1CCC1C2C(=O)C[C@@]2(C)C1CC[C@]2(O)C(=O)CO ; C[C@]12C=CC(=O)C=C1CCC1C2[C@@H](O)C[C@@]2(C)C1CC[C@]2(O)C(=O)CO]

11. The process according to anyone of claims 3-10, wherein said compound of formula (2) is administered to said bacterial culture in a concentration comprised between 0.5 grams/Liter and 10 grams/Liter, preferably comprised between 1 gram/Liter and 5 grams/Liter.

12. The process according to anyone of claims 3-11 for the obtainment of a compound of formula (1A) wherein said compound of formula (2) is selected from: cortisone, cortisol, prednisone and prednisolone.

13. The process according to anyone of claims 3-11 for the obtainment of a compound of formula (1B) wherein said compound of formula (2) is selected from: cortisone and prednisone.

14. Compound of claim 1 or 2 for use as a medicament.

15. Compound of claim 1 or 2 for use in the treatment of inflammatory diseases and/or autoimmune diseases.

16. Compound for the use of claim 15, wherein said inflammatory and/or autoimmune disease is selected from the group consisting of: asthma, dermatitis, intestinal inflammatory diseases, rheumatoid arthritis and multiple sclerosis.

17. Compound of claim 1 or 2 for use in the treatment of diseases wherein a joined anti-inflammatory and anti-oxidant activity is needed.

18. Compound of claim 1 or 2 for use in the treatment of diseases due to allergic reactions or in the prevention of transplant rejection.

19. Compound of claim 1 or 2 for use in the treatment of neurodegenerative diseases, such as Alzheimer's disease and Parkinson's disease.

20. Use of the compound of claim 1 or 2 for the cosmetic treatment of the skin, in particular to prevent skin ageing.

21. A pharmaceutical composition comprising at least one compound of claim 1 or 2 together with at least one suitable pharmaceutically acceptable vehicle.

22. The composition of claim 21 which is in an aqueous formulation.
